(19) United States
(12) Patent Application Publication
(10) Pub. No.: US 2008/0014263 A1
(43) Pub. Date: Jan. 17, 2008

(54) Amorphous eprosartan mesylate and process for the preparation thereof

(75) Inventors: Anthony Melvin Crasto, Thane (w) (IN); Samir Jaivant Naik, Thane (IN); Narendra Sriram Joshi, Navi Mumbai (IN); Mubeen Khan, Navi Mumbai (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/879,334

(22) Filed: Jul. 17, 2007

(30) Foreign Application Priority Data
Jul. 17, 2006 (IN) 1134/MUM/2006

(51) Int. Cl. A61K 9/20; A61K 31/4178; A61P 9/12; C07D 233/00; A61K 9/14

(52) U.S. Cl. 424/464; 424/489; 514/397; 548/315.1

(57) ABSTRACT

Eprosartan mesylate substantially in an amorphous form is disclosed. A composition comprising eprosartan mesylate in a solid form, wherein at least about 80% by weight of the solid eprosartan mesylate is in an amorphous form is also disclosed. The present invention also provides a process for preparing eprosartan mesylate substantially in an amorphous form by providing a solution of eprosartan mesylate in one or more solvents capable of dissolving the eprosartan mesylate and substantially removing the solvent from the solution.

### PRIORITY

This application claims the benefit under 35 U.S.C. § 119 to Indian Provisional Application No. 1134/MUM/2006, filed on Jul. 17, 2006, the contents of which are incorporated by reference herein.

### BACKGROUND OF THE INVENTION

1. Technical Field

The present invention generally relates to substantially amorphous eprosartan mesylate and process for the preparation thereof. More particularly, the present invention generally relates to a composition comprising eprosartan mesylate in a solid form, wherein at least 80% by weight of the solid eprosartan mesylate is in amorphous form.

2. Description of the Related Art

TEVETEN® (eprosartan mesylate) tablet is a non-biphenyl non-tetrazole angiotensin II receptor (AT₁) antagonist. A selective non-peptide molecule, TEVETEN® tablets are chemically described as the monomethanesulphonate of (E)-2-butyl-1-(p-carboxybenzyl)-α-2- thienylmethylimidazole-5-acrylic acid. Eprosartan mesylate is represented by the structural of Formula I.

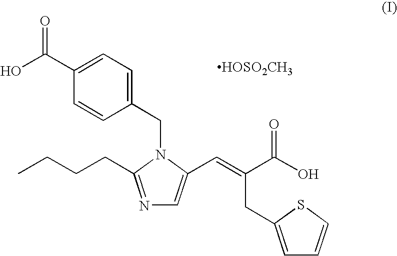

The empirical formula of eprosartan mesylate is C₂₃H₂₄N₂O₄S.CH₄O₃S and has a molecular weight of 520.625. Eprosartan mesylate is a white to off-white free-flowing crystalline powder that is insoluble in water, freely soluble in ethanol, and melts between 248° C. and 250° C.

TEVETEN® Tablets are available as aqueous film-coated tablets containing eprosartan mesylate equivalent to 400 mg or 600 mg Eprosartan zwitterions (pink, scored Tiltab®, oval or white capsule-shaped tablets, respectively).

U.S. Pat. No. 5,185,351, herein incorporated by reference, discloses eprosartan mesylate and processes for its preparation.

Polymorphism is the occurrence of different crystalline forms of a single compound and it is a property of some compounds and complexes. Polymorphs are distinct solids sharing the same molecular formula. A single compound may give rise to a variety of polymorphic forms, and each form may have a different and distinct physical properties, such as solubility profiles, melting point temperatures and/or x-ray diffraction peaks. Polymorphic forms of a compound can be distinguished in a laboratory by X-ray diffraction spectroscopy and by other methods such as, infrared spectrometry.

Since the solubility of each polymorph may vary, identifying the existence of pharmaceutical polymorphs is essential for providing pharmaceuticals with predicable solubility profiles. It is desirable to investigate and identify the solid state forms of a drug, including the polymorphic forms, and to determine the stability, dissolution and flow properties of each polymorphic form. Polymorphic forms of a drug or active pharmaceutical ingredient (“API”) can be administered alone or can be formulated with other excipients as a drug product (also known as the final or finished dosage form). It is also well known in the pharmaceutical art that the polymorphic form can affect the physical properties of the drug or API, for example, increasing or decreasing the solubility, stability, flowability, tractability and compressibility of drug substances, as well as the safety and efficacy of drug products.

Furthermore, amorphous materials do not exhibit the three-dimensional long-range order found in crystalline materials, but is structurally more similar to liquids where the arrangement of molecules is random. Amorphous solids do not give a definitive x-ray diffraction pattern (XRD). In addition, amorphous solids do not give rise to a melting point and tend to liquefy at some point beyond the glass transition point. Because amorphous solids do not have lattice energy, they usually dissolve in a solvent more rapidly and consequently may provide rapid bioavailability. Furthermore, amorphous forms of a drug may offer significant advantages over crystalline forms of the same drug in solid dosage form manufacture process such as compressibility, economically or environmentally suitable solvents or process, or higher purity or yield of the desired product.

Accordingly, there remains a need to reproducibly obtain amorphous eprosartan mesylate of similar quality for use in a pharmaceutical preparation.

### SUMMARY OF THE INVENTION

In accordance with one embodiment of the present invention, eprosartan mesylate substantially in an amorphous form is provided.

In accordance with a second embodiment of the present invention, a composition is provided comprising eprosartan mesylate in a solid form, wherein at least about 80% by weight of the solid eprosartan mesylate is in an amorphous form.

In accordance with a third embodiment of the present invention, a process for preparing eprosartan mesylate substantially in an amorphous form is provided, the process comprising the steps of:
(a) providing a solution of eprosartan mesylate in one or more solvents capable of dissolving the eprosartan mesylate;
(b) optionally, filtering the solvent solution to substantially remove any extraneous matter; and
(c) substantially removing the solvent from the solution to provide eprosartan mesylate substantially in an amorphous form.

### DETAILED DESCRIPTION OF THE INVENTION

The present invention provides eprosartan mesylate substantially in an amorphous form. The amorphous eprosartan mesylate can be prepared by a process involving (a) providing a solution of eprosartan mesylate in one or more solvents capable of dissolving the eprosartan mesylate; (b) optionally, filtering the solvent solution to remove any extraneous matter; and (c) substantially removing the solvent from the solution to provide eprosartan mesylate substantially in an amorphous form.

Step (a) of the process of the present invention includes dissolving any form of eprosartan mesylate in a suitable solvent or obtaining an existing solution from a previous processing step. Suitable solvents include, but are not limited to, an alcohol, ketone, ester, ether, nitrile, acid, water and mixtures thereof. In one embodiment, the solvent is selected from the group consisting of an alcoholic solvent having from 1 to 6 carbon atoms, such as methanol, ethanol and the like, aromatic hydrocarbon solvent, such as xylene, toluene and the like, non-aromatic hydrocarbon solvents, such as hexane, and mixtures thereof. The dissolution can be carried out at a temperature ranging from about 0° C. to about 150° C. and preferably at room temperature.

The clear solution may optionally be filtered to remove any extraneous matter present in the solution using any standard filtration techniques known in the art.

Step (c) of the process of the present invention can be carried out by, for example, substantially complete evaporation of the solvent, concentrating the solution, cooling to obtain amorphous form and filtering the solid under inert atmosphere. Alternatively, the solvent may also be removed by evaporation. Evaporation can be achieved at sub-zero temperatures by the lyophilisation or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated

A preferred method to remove the solvent involves spray-drying, in which a solution of eprosartan mesylate is sprayed into the spray drier at the flow rate ranging from about 10 to about 300 ml/hr, and preferably about 40 to about 200 ml/hr. The air inlet temperature to the spray drier used may range from about 25° C. to about 150° C., and preferably from about 60° C. to about 110° C. and the outlet air temperature used may range from about 30° C. to about 90° C. Of course, specific conditions will vary somewhat for spray drying using different equipment configurations. The solid residue obtained after the solvent removal is isolated and, if desired, can be dried further using conventional methods. The advantages of the process include simplicity, eco-friendliness and suitability for commercial use.

Another preferred method is vertical agitated thin-film drying (or evaporation). Agitated thin film evaporation technology involves separating the volatile component using indirect heat transfer coupled with mechanical agitation of the flowing film under controlled condition. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

The substantially pure amorphous eprosartan mesylate obtained by the above processes may be further dried in, for example, Vacuum Tray Dryer, Rotocon Vacuum Dryer, Vacuum Paddle Dryer or pilot plant Rota vapor, to further lower residual solvents.

Another aspect of the present invention provides eprosartan mesylate substantially in an amorphous form, having a chemical purity of at least about 96% or more as measure by HPLC, preferably at least about 99% or more, and more preferably at least about 99.5% or more.

Yet another aspect of the present invention is directed to a pharmaceutical composition containing at least the novel eprosartan mesylate in substantially amorphous form of the present invention, wherein the D₅₀ and D₉₀ particle size of the unformulated eprosartan mesylate used as starting material is less than about 300 microns, preferably less than about 200 microns, more preferably less than about 150 microns, still more preferably less than about 50 microns and most preferably less than about 10 microns. The particle sizes of the novel eprosartan mesylate substantially in an amorphous form of the present invention can be obtained by, for example, any milling, grinding, micronizing or other particle size reduction method known in the art to bring the solid state eprosartan mesylate substantially in an amorphous form of the present invention into any of the foregoing desired particle size range.

It is particularly advantageous when formulating eprosartan mesylate for commercial use, that amorphous eprosartan mesylate is prepared in a relatively reduced size for high drug load tablets. A wet milling procedure can be used to produce amorphous eprosartan mesylate of reduced size having a surface modifier adsorbed on the surface thereof in an amount sufficient to maintain an average particle size less than about 1000 nm, preferably less than about 500 nm, more preferably less than about 400 nm and most preferably less than about 250 nm.

In addition to X-ray powder diffraction, amorphous eprosartan mesylate, or the presence of some amorphous eprosartan mesylate, can be distinguished from crystalline eprosartan mesylate, using, for example, Raman spectroscopy, solution calorimetry, differential scanning calorimetry, solid state nuclear magnetic resonance spectra (ssNMR) or infra-red spectroscopy. Each of these techniques is well established in the art. Amorphous eprosartan mesylate can also be identified based on the morphology of the particles seen under an electron microscope.

Unless defined otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described.

For purposes of the present invention, the following terms are defined below.

The term “composition” includes, but is not limited to, a powder, a suspension, an emulsion and/or mixtures thereof. The term “composition” is intended to encompass a product containing the specified ingredients in the specified amounts, as well as any product, which results, directly or indirectly, from combination of the specified ingredients in the specified amounts. A “composition” may contain a single compound or a mixture of compounds.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, additional active ingredient(s), and pharmaceutically acceptable excipients.

The term “excipient” shall be understood to mean a component of a pharmaceutical product that is not an active ingredient, such as a filler, diluent, carrier, and the like. Excipients that are useful in preparing a pharmaceutical composition are generally safe, non-toxic and neither biologically nor otherwise undesirable, and are acceptable for veterinary use as; well as human pharmaceutical use. A “pharmaceutically acceptable excipient” as used in the specification and claims includes one or more such excipients.

The term “isolating” is used to indicate separation of the compound being isolated regardless of the purity of the isolated compound from any unwanted substance which presents with the compound as a mixture. Thus, degree of the purity of the isolated or separated compound does not affect the status of isolating”.

It is known that different solid forms of the same drug may have substantial differences in certain pharmaceutically important properties such as dissolution characteristics and bioavailability, as well as stability of the drug. Furthermore, different physical forms may have different particle size, hardness and glass transition temperatures.

The invention also relates to a composition of solid eprosartan mesylate wherein at least about 80% of the total weight of eprosartan mesylate is in the amorphous form. A preferred form of this composition, the solid eprosartan mesylate is suitable for use as a bulk active ingredient in formulating pharmaceutical products. The remainder of the solid eprosartan mesylate in the composition, i.e., about 20% or less of the total weight of eprosartan mesylate, may be other forms of eprosartan mesylate, e.g., crystalline forms. In an embodiment of the invention, the composition may include at least about 95% of the amorphous form of eprosartan mesylate with respect to total weight of the solid eprosartan mesylate in the composition. In another embodiment of the invention, the composition may include at least about 99% of the amorphous form of eprosartan mesylate with respect to total weight of the solid eprosartan mesylate in the composition.

Furthermore, eprosartan mesylate substantially in an amorphous form is likely to be much more soluble than crystalline eprosartan mesylate because the former lacks lattice energy, providing another means of discriminating between the crystalline and amorphous eprosartan mesylate forms, or detecting an amount of amorphous form within a eprosartan mesylate preparation.

Another embodiment of the present invention provides pharmaceutical compositions containing the novel eprosartan mesylate substantially in an amorphous form, which can be formulated with a one or more pharmaceutically acceptable carriers, also known as excipients, which ordinarily lack pharmaceutical activity, but have various useful properties which may, for example, enhance the stability, sterility, bioavailability, and ease of formulation of a pharmaceutical composition. These carriers are pharmaceutically acceptable, meaning that they are not harmful to humans or animals when taken appropriately and are compatible with the other ingredients in a given formulation. The carriers may be solid, semi-solid, or liquid, and may be formulated with the compound in bulk. The resulting mixture may be manufactured in the form of a unit-dose formulation (i.e., a physically discrete unit containing a specific amount of active ingredient) such as a tablet or capsule.

Generally, the pharmaceutical compositions of the present invention may be prepared by uniformly admixing the active ingredient with liquid or solid carriers and then shaping the product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed. A tablet may be prepared by direct compression, wet granulation, or molding, of the active ingredient(s) with a carrier and other excipients in a manner known to those skilled in the art. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made on a suitable machine. A mixture of the powdered compound moistened with an inert liquid diluent is suitable in the case of oral solid dosage forms (e.g., powders, capsules, and tablets). If desired, tablets may be coated by standard techniques. The compounds of this invention may be formulated into typical disintegrating tablets, or into controlled or extended release dosage forms. The amount of active ingredient included in a unit dosage form depends on the type of formulation that is formulated. A pharmaceutical composition of the invention will generally include about 0.1% by weight to about 99% by weight of active ingredient, preferably about 1% by weight to about 50% by weight.

Suitable carriers include but are not limited to fillers, binders, lubricants, inert diluents, surface active/dispersing agents, flavorants, antioxidants, bulking and granulating agents, adsorbants, preservatives, emulsifiers, suspending and wetting agents, glidants, disintegrants, buffers and preadjusting agents, colorants and the like and mixtures thereof. Examples of carriers include celluloses, modified celluloses, cyclodextrins, starches, oils, polyols, sugar alcohols and sugars, and others. For liquid formulations sugar, sugar alcohols, ethanol, water, glycerol, and polyalkylene glycols are particularly suitable, and may also be used in solid formulations. Cyclodextrins may be particularly useful for increasing bioavailability. Formulations for oral administration may optionally include enteric coatings known in the art to prevent degradation of the formulation in the stomach and provide release of the drug in the small intestine. One example of a pharmaceutical tablet of the present invention includes amorphous eprosartan mesylate and may include, as inactive ingredients, hypromellose 2910, lactose monohydrate, magnesium stearate, microcrystalline cellulose, polyethylene glycol 3000, sodium starch glycolate, titanium dioxide, triacetin and one or more of synthetic red and yellow iron oxides and talc.

The novel eprosartan mesylate substantially in an amorphous form may also be administered via fast dispersing or fast dissolving dosage forms or in the form of high energy dispersion or as coated particles. Suitable pharmaceutical composition of the invention may be in coated or uncoated form as desired.

Tabletting compositions may have few or many components depending upon the tabletting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

Capsule dosages will contain the solid composition within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric coating.

Actual dosage levels of the eprosartan mesylate substantially in an amorphous form of the present invention may be varied to obtain an amount of the amorphous form of eprosartan mesylate that is effective to obtain a desired therapeutic response for a particular composition and method of administration for treatment of a mammal. The selected dosage level therefore depends upon such factors as, for example, the desired therapeutic effect, the route of administration, the desired duration of treatment, and other factors. The total daily dose of the eprosartan mesylate substantially in an amorphous form thereof of the present invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc. A pharmaceutical composition of the invention will generally include about 0.1% by weight to about 99% by weight of active ingredient, and preferably about 1% by weight to 50% by weight.

The following examples are provided to enable one skilled in the art to practice the invention and are merely illustrative of the invention. The examples should not be read as limiting the scope of the invention as defined in the features and advantages.

### EXAMPLE 1

### Preparation of Amorphous Eprosartan Mesylate by Spray Drying

Eprosartan mesylate is dissolved in an alcoholic solvent such as methanol (40 volumes) in a round bottom flask. The solution is filtered through a filtration medium or filter aid to remove any extraneous matter. The filtrate is then spray dried at 40 to 45° C. until the solvent is completely removed. The solid obtained is collected and dried under high vacuum at a suitable temperature and a free flowing amorphous solid is obtained.

### EXAMPLE 2

### Preparation of Amorphous Eprosartan Mesylate by Vertical Agitated Thin Film Drying

Eprosartan mesylate and an alcoholic solvent such as methanol (40 volumes) are charged into a clean and dry round bottom flask and stirred for about 30 minutes. The solution is filtered through a filtration medium or filter aid to remove any extraneous matter. The filtrate is subjected to agitated thin film drying with a feed rate of about 5 L/hour, under a reduced pressure of about 5-20 torr and a jacket temperature of about 35-40° C. The solid obtained is collected and dried under high vacuum at a suitable temperature and a free flowing amorphous solid is obtained.

### EXAMPLE 3

### Preparation of Amorphous Eprosartan Mesylate by Lyophilisation

Eprosartan mesylate is dissolved in an alcoholic solvent such as methanol (40 volumes) at room temperature. The solution is filtered through a filtration medium or filter aid to remove any extraneous matter. The filtrate is subjected to lyophilisation until a free flowing amorphous solid is obtained.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. Eprosartan mesylate in an amorphous form.

2. A composition comprising eprosartan mesylate in a solid form, wherein at least about 80% by weight of the solid is eprosartan mesylate in an amorphous form.

3. The composition of claim 2, wherein the amorphous eprosartan mesylate has a chemical purity of at least about 96%.

4. The composition of claim 2, wherein the amorphous eprosartan mesylate has a chemical purity of at least about 99%.

5. The composition of claim 2, wherein the amorphous eprosartan mesylate has a chemical purity of at least about 99.5%.

6. The composition of claim 2, wherein the eprosartan mesylate has a D₅₀ particle size of less than about 300 microns.

7. The composition of claim 2, wherein the eprosartan mesylate has a D₅₀ particle size of less than about 50 microns.

8. The composition of claim 2, wherein the eprosartan mesylate has a D₅₀ particle size less than about 10 microns.

9. The composition of claim 2, wherein the eprosartan mesylate has a D₉₀ particle size of less than about 300 microns.

10. The composition of claim 2, wherein the eprosartan mesylate has a D₉₀ particle size of less than about 50 microns.

11. The composition of claim 2, wherein the eprosartan mesylate has a D₉₀ particle size less than about 10 microns.

12. The composition of claim 2, wherein the amorphous eprosartan mesylate has an average particle size less than about 500 nm.

13. The composition of claim 2, wherein the amorphous eprosartan mesylate has an average particle size less than about 400 nm.

14. The composition of claim 2, wherein the amorphous eprosartan mesylate has an average particle size less than about 250 nm.

15. The composition of claim 12, in the form of a high drug load tablet.

16. A pharmaceutical composition comprising a therapeutically effective amount of the eprosartan mesylate in an amorphous form of claim 1 and at least one pharmaceutically acceptable excipient.

17. A pharmaceutical composition comprising a therapeutically effective amount of the composition of claim 2 and at least one pharmaceutically acceptable excipient.

18. A process for preparing eprosartan mesylate substantially in an amorphous form, the process comprising:
(a) providing a solution of eprosartan mesylate in one or more solvents capable of dissolving the eprosartan mesylate; and
(b) substantially removing the solvent from the solution to provide amorphous eprosartan mesylate.

19. The process of claim 18, wherein step (b) comprises spray drying.

20. The process of claim 18, wherein the solvent is selected from the group consisting of an alcoholic solvent having from 1 to 6 carbon atoms, aromatic hydrocarbon solvent, non-aromatic hydrocarbon solvents and mixtures thereof.
